(19) United States
(12) Patent Application Publication
(10) Pub. No.: US 2017/0176581 A1
(43) Pub. Date: Jun. 22, 2017

(54) ULTRASONIC IMAGE APPARATUS AND CONTROL METHOD THEREOF

(71) Applicant: Samsung Electronics Co., Ltd., Suwon-si (KR)

(72) Inventor: Yeon-woo KU, Suwon-si (KR)

(21) Appl. No.: 15/358,772

(22) Filed: Nov. 22, 2016

(30) Foreign Application Priority Data
Dec. 21, 2015 (KR) 10-2015-0186732

(51) Int. Cl. G01S 7/52; A61B 8/00; A61B 8/14; G01S 7/56; G01S 15/02

(52) U.S. Cl. CPC G01S 7/52004; G01S 7/56; G01S 15/02; A61B 8/145; A61B 8/4483; A61B 8/58

(57) ABSTRACT

An ultrasonic image apparatus is provided. The ultrasonic image apparatus includes a plurality of transducers configured to output an ultrasonic signal and receive a feedback signal, a driver comprising a plurality of driving circuits respectively connected to the plurality of transducers, each driving circuit configured to output the ultrasonic signal and receive the feedback signal, a detection circuit respectively corresponding to the plurality of driving circuits, the detection circuit configured to detect whether any of the plurality of transducers is defective, and at least one processor configured to control the driving circuit to stop operations corresponding to at least one defective transducer among the plurality of transducers based on a defect detection result from the detection circuit with regard to the plurality of transducers.

### CROSS-REFERENCE TO RELATED APPLICATION(S)

This application claims the benefit under 35 U.S.C. §119(a) of a Korean patent application filed on Dec. 21, 2015 in the Korean Intellectual Property Office and assigned Serial number 10-2015-0186732, the entire disclosure of which is hereby incorporated by reference.

### TECHNICAL FIELD

The present disclosure relates to an ultrasonic image apparatus and a control method thereof. More particularly, the present disclosure relates to an ultrasonic image apparatus and a control method thereof, which can ensure the reliability of a transducer used in taking an ultrasonic image.

### BACKGROUND

With the development of electronic technology, the application of electronic technology has been gradually extended into the field of medical and health care. In particular, there has been the creation and use of an ultrasonic device as a device for medical and clinical diagnosis.

The ultrasonic device typically employs a transducer to capture an image of a diagnostic region. In the case of a matrix-array transducer used for taking a finer ultrasonic image, the number of arrays may be up to 100 times greater than that of a general transducer and therefore, the transducer is more likely to be defective due to a manufacturing process, a process of connecting with an application-specific integrated circuit (ASIC), or similar complicated processes, in regard to the larger number of arrays.

If the transducer is defective, it causes problems of not only low performance, but also overheating that endangers safety. Such overheating generally occurs when the transducer is broken and experiences a short circuit, thereby damaging the entire ultrasonic device. Further, overheating may lead to safety problems since the ultrasonic device is a medical apparatus typically used directly touching a skin surface.

Therefore, there is a need for detecting whether an individual transducer is detective in early stages, and prior to use of the matrix-array transducer such as on a skin surface.

The above information is presented as background information only, and to assist with an understanding of the present disclosure. No determination has been made, and no assertion is made, as to whether any of the above might be applicable as prior art with regard to the present disclosure.

### SUMMARY

Aspects of the present disclosure are provided to address at least the above-mentioned problems and/or disadvantages, and to provide at least the advantages described below. Accordingly, an aspect of the present disclosure is to provide an ultrasonic image apparatus and a control method thereof, which can detect whether any of a plurality of transducers used therein is defective.

Another aspect of the present disclosure is to provide an ultrasonic image apparatus and a control method thereof, which can selectively inactivate a defective transducer among the plurality of transducers used therein to ensure safety.

Another aspect of the present disclosure is to provide an ultrasonic image apparatus and a control method thereof, which can output a common voltage to a defective transducer among the plurality of transducers used therein, thereby preventing decreased image quality.

In accordance with an aspect of the present disclosure, an ultrasonic image apparatus is provided. The ultrasonic image apparatus includes a plurality of transducers configured to output an ultrasonic signal and receive a feedback signal, a driver comprising a plurality of driving circuits respectively connected to the plurality of transducers, each driving circuit configured to output the ultrasonic signal and receive the feedback signal, a detection circuit respectively corresponding to the plurality of driving circuits, the detection circuit configured to detect whether any of the plurality of transducers is defective, and at least one processor configured to control the driving circuit to stop operations corresponding to at least one defective transducer among the plurality of transducers based on a defect detection result from the detection circuit with regard to the plurality of transducers.

According to an embodiment, it is possible to ensure reliability of the transducer since there is a determination whether any of the plurality of transducers is defective before the ultrasonic measurement. Further, an application specific integrated circuit (ASIC) corresponding to a defective transducer among the plurality of transducers is inactivated before the ultrasonic measurement, thereby ensuring safety.

The detection circuit may detect whether any of the plurality of transducers is defective based on whether a voltage passed through one or more of the plurality of transducers is out of a preset range. Thus, it is possible to determine whether any of the plurality of transducers is defective in accordance with whether there is a voltage drop.

The detection circuit may include a low-voltage current source and a comparator. Thus, it is possible to detect whether any of the plurality of transducers is defective at a low voltage, and it is possible to detect a defect in accordance with whether there is an abnormal voltage drop as compared with a preset voltage.

The at least one processor may be configured to control the detection circuit to stop operations corresponding to a transducer of which a defect detection is completed, among the plurality of transducers. Thus, the detection circuit is inactivated after detecting whether any of the plurality of transducers is defective.

The ultrasonic image apparatus may further include a voltage setting circuit corresponding to each of the plurality of driving circuits, wherein the voltage setting circuit is configured to output a voltage having a preset level corresponding to at least one transducer detected as defective by the detection circuit. Thus, a common voltage is output to the defective transducer, thereby preventing deterioration of image quality.

The voltage setting circuit may include a switch for selectively controlling an output of a voltage having a preset level. Thus, it is possible to control the switch to output the common voltage to the defective transducer.

The at least one processor may be configured to control a supply of power based on a proportion of driving circuits stopped in accordance with defect detection results from the detection circuit, among the plurality of driving circuits. Thus, if a proportion of transducers detected as defective among the plurality of transducers is high, the apparatus is automatically turned off.

The ultrasonic image apparatus may further include a display, and the at least one processor may be configured to control the display to display a defect detection result from the detection circuit with regard to each of the plurality of transducers. Thus, a defect detection result in each of the plurality of transducers is displayed on a screen so that a user can be informed of the result.

The ultrasonic image apparatus may further include a communicator for connecting with a display apparatus, wherein the at least one processor may be configured to control the communicator to transmit a defect detection result from the detection circuit with regard to each of the plurality of transducers to the display apparatus. Thus, a defect detection result in each of the plurality of transducers is transmitted to a connected display apparatus so that a user can be informed of the result.

The detection circuit may be provided as a single circuit corresponding to the plurality of driving circuits, and may connect with each of the plurality of driving circuits in sequence to detect whether the plurality of transducers is defective. Thus, a single detection circuit provided in common with regard to the plurality of transducers is used to sequentially detect whether any of the plurality of transducers is defective.

Any one or more of the plurality of driving circuits itself may be used to detect whether any of the plurality of transducers is defective. Thus, without any separate detection circuit, the plurality of ASICs corresponding to the plurality of transducers is provided with an element for detecting a defect, thereby detecting whether any transducer is defective.

The plurality of driving circuits may include a comparator for detecting whether any of the plurality of transducers is defective. Thus, if any separate detection circuit is not provided, a defect can be detected by the ASIC in accordance with whether there is a voltage drop as compared with a preset voltage with respect to each transducer.

In accordance with another aspect of the present disclosure, a method of controlling an ultrasonic image apparatus is provided. The method includes detecting whether any of a plurality of transducers is defective through a detection circuit provided in each of a plurality of driving circuits respectively connected to the plurality of transducers, each driving circuit configured to output an ultrasonic signal and receive a feedback signal, and stopping an operation of a driving circuit corresponding to at least one defective transducer among the plurality of transducers based on a defect detection result from the detection circuit with regard to the plurality of transducers.

According to an embodiment, it is possible to ensure reliability of the transducer since there is a determination whether any of the plurality of transducers is defective before ultrasonic measurement. Further, an ASIC corresponding to a defective transducer among the plurality of transducers is inactivated before ultrasonic measurement, thereby ensuring safety.

The detecting of whether any of a plurality of transducers is defective may include detecting whether any of the plurality of transducers is defective based on whether a voltage passed through one or more of the plurality of transducers is out of a preset range. Thus, it is possible to determine whether any of the plurality of transducers is defective in accordance with whether there is a voltage drop.

The detection circuit may include a low-voltage current source and a comparator. Thus, it is possible to detect whether any of the plurality of transducers is defective at a low voltage, and it is possible to detect a defect in accordance with whether there is an abnormal voltage drop as compared with a preset voltage.

The method may further include stopping an operation of the detection circuit corresponding to a transducer of which a defect detection is completed, among the plurality of transducers. Thus, the detection circuit is inactivated after detecting whether any of the plurality of transducers is defective.

The stopping of the operation of the driving circuit may include outputting a voltage having a preset level corresponding to at least one transducer detected as defective in the detection circuit, by a voltage setting circuit provided corresponding to each of the plurality of driving circuits. Thus, a common voltage is output to the defective transducer, thereby preventing deterioration of image quality.

The voltage setting circuit may include a switch for selectively controlling an output of a voltage having a preset level. Thus, it is possible to control the switch to output the common voltage to the defective transducer.

The method may further include controlling a supply of power based on a proportion of driving circuits stopped in accordance with defect detection results from the detection circuit, among the plurality of driving circuits. Thus, if a proportion of transducers detected as defective among the plurality of transducers is high, the apparatus is automatically turned off.

The method may further include displaying a defect detection result from the detection circuit with regard to each of the plurality of transducers. Thus, a defect detection result in each of the plurality of transducers is displayed on a screen so that a user can be informed of the result.

The method may further include transmitting a defect detection result from the detection circuit with regard to each of the plurality of transducers to a display apparatus. Thus, a defect detection result in each of the plurality of transducers is transmitted to a connected display apparatus so that a user can be informed of the result.

The method may further include detecting whether the plurality of transducers is defective by connecting the detection circuit with each of the plurality of driving circuits in sequence, wherein the detection circuit is provided as a single circuit corresponding to the plurality of driving circuits. Thus, a single detection circuit provided in common with regard to the plurality of transducers is used to sequentially detect whether any of the plurality of transducers is defective.

The method may further include detecting whether any of the plurality of transducers is defective by using one or more of the plurality of driving circuits itself. Thus, without any separate detection circuit, the plurality of ASICs corresponding to the plurality of transducers is provided with an element for detecting a defect, thereby detecting whether any transducer is defective.

The plurality of driving circuits may include a comparator for detecting whether any of the plurality of transducers is defective. Thus, if any separate detection circuit is not provided, a defect can be detected by the ASIC in accordance with whether there is a voltage drop as compared with a preset voltage with respect to each transducer.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and/or other aspects, features and advantages of certain embodiments of the present disclosure will be more apparent from the following description taken in conjunction with the accompanying drawings, in which:

FIG. 1 is a block diagram of an ultrasonic image apparatus according to an embodiment of the present disclosure;

FIG. 2 is a block diagram of an ultrasonic image apparatus according to an embodiment of the present disclosure;

FIG. 3 is a block diagram of an ultrasonic image apparatus according to an embodiment of the present disclosure;

FIG. 4 illustrates a structure of an application-specific integrated circuit (ASIC) with respect to an individual transducer according to an embodiment of the present disclosure;

FIG. 5 illustrates a structure of an ASIC with respect to an individual transducer according to an embodiment of the present disclosure;

FIG. 6 illustrates a structure of an ASIC with respect to an individual transducer according to an embodiment of the present disclosure;

FIG. 7 illustrates an operation for detecting a defect in a one-dimensional matrix-array transducer according to an embodiment of the present disclosure;

FIG. 8 illustrates an operation for detecting a defect in a two-dimensional matrix-array transducer according to an embodiment of the present disclosure;

FIG. 9 shows results from detecting a defect in a one-dimensional matrix-array transducer according to an embodiment of the present disclosure;

FIG. 10 shows results from detecting a defect in a two-dimensional matrix-array transducer according to an embodiment of the present disclosure;

FIG. 11 illustrates an operation for detecting a defect in each of the plurality of transducers through a common defect detecting circuit according to an embodiment of the present disclosure;

FIG. 12 illustrates a case where defective transducers are widely dispersed according to an embodiment of the present disclosure;

FIG. 13 illustrates a case where defective transducers are densely concentrated in a certain area according to an embodiment of the present disclosure;

FIG. 14 illustrates a defective piezoelectric (PZT) transducer according to an embodiment of the present disclosure;

FIG. 15 is a flowchart of a process of protecting a plurality of transducers according to an embodiment of the present disclosure;

FIG. 16 is a flowchart of a defect detecting process with regard to an individual transducer according to an embodiment of the present disclosure;

FIG. 17 is a flowchart of a method of controlling an ultrasonic image apparatus according to an embodiment of the present disclosure; and

FIG. 18 is a flowchart of a method of controlling an ultrasonic image apparatus according to an embodiment of the present disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding, but these are to be regarded as merely exemplary. Accordingly, those of ordinary skill in the art will recognize that various changes and modifications of the various embodiments described herein can be made without departing from the spirit and scope of the present disclosure. In addition, descriptions of well-known functions and constructions may be omitted for clarity and conciseness.

The terms and words used in the following description and claims are not limited to the bibliographical meanings, but are merely used to enable a clear and consistent understanding of the present disclosure. Accordingly, it should be apparent to those skilled in the art that the following description of various embodiments of the present disclosure is provided for illustration purposes only, and not for the purpose of limiting the present disclosure as defined by the appended claims and their equivalents.

It is to be understood that the singular forms “a,” “an,” and “the”, include plural referents unless the context clearly dictates otherwise. Thus, for example, reference to “a component surface” includes reference to one or more of such surfaces.

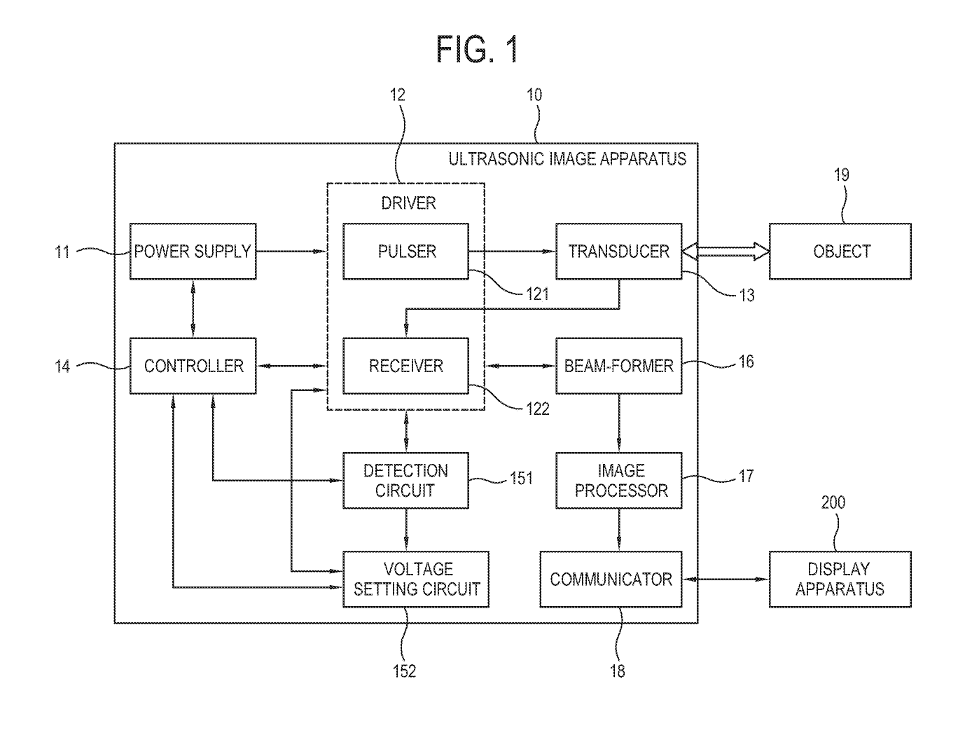

Below, elements included in an ultrasonic image apparatus according to an embodiment will be described in detail starting with reference to FIG. 1.

FIG. 1 is a block diagram of an ultrasonic image apparatus according to an embodiment of the present disclosure.

Referring to FIG. 1, an ultrasonic image apparatus 10 according to an embodiment includes a power supply 11, a driver 12, a plurality of transducers 13, a controller 14, a detection circuit 151, a voltage setting circuit 152, a beam-former 16, an image processor 17 and a communicator 18. The driver 12 includes a pulser 121 and a receiver 122. According to an embodiment, the ultrasonic image apparatus 10 may communicate with an external display apparatus 200 or other device or system through the communicator 18.

According to an embodiment, the ultrasonic image apparatus 10 may further include a light source (not shown). For example, the light source may include at least one light source that emits light having a certain wavelength. Alternatively, the light source may include a plurality of light sources that emit light of different wavelengths. The wavelength of the light emitted from the light source may be selected in consideration of a target within an object. Such a light source may be achieved by using a semiconductor laser, a light emitting diode (LED), a solid laser, a gas laser, optical fiber, or some combination thereof.

According to an embodiment, the elements included in the ultrasonic image apparatus 10 are not limited to the foregoing description, but may include other additional elements or elements in various combinations, or may exclude some elements.

According to an embodiment, the ultrasonic image apparatus 10 refers to a medical apparatus which transmits an ultrasonic signal from a body surface of an object toward predetermined tissue inside the body, and takes a tomogram of the tissue inside the body or an image of blood flow based on information from an ultrasonic feedback signal reflected from the tissue inside the body. In detail, when the ultrasonic image apparatus 10 transmits ultrasonic waves having frequencies of several MHz to hundreds of MHz to a certain region inside a patient's body, the ultrasonic waves are partially reflected from layers between various tissues. The ultrasonic waves are also detectably reflected from anatomical objects inside the body, which are different in density, for example, from blood cells in blood plasma, small tissues in organs, and so forth.

According to an embodiment, the ultrasonic image apparatus 10 may be achieved in various forms. For example, the ultrasonic image apparatus 10 may be a mobile terminal or a stationary terminal. If the ultrasonic image apparatus 10 is achieved by using a mobile terminal, an ultrasonic image generated by the ultrasonic image apparatus 10 is transmitted to the connected display apparatus 200 so that the display apparatus can display the ultrasonic image. The connected display apparatus 200 may be achieved by using for example, a smart phone, a tablet computer, a smart television (TV), a desktop computer, a laptop computer, a personal digital assistant (PDA), and the like.

According to an embodiment, the ultrasonic image apparatus 10 may also exchange medical image data with a hospital server or other medial apparatuses in a hospital, connected through a picture archiving and communication system (PACS). Further, the ultrasonic image apparatus 10 may perform data communication with a server or the like, in accordance with digital imaging and communications in medicine (DICOM).

According to an embodiment, the display apparatus 200 connected to the ultrasonic image apparatus 10 may include a touch screen or similar feature. The touch screen may be configured to detect a position of a touch input, an area of a touch input, and the touch input. Further, the touch screen may be configured to detect not only a real touch or touches, but also a proximity or hovering touch or touches. A real touch describes a touch where the touch screen is actually touched with a user's body (e.g. a finger) or a touch pen provided as a touch tool (e.g. a pointing device, a stylus, a haptic pen, an electronic pen, or the like). A proximity or hovering touch describes a touch where a user's body or a touch tool does not actually touch the touch screen, but is within a predetermined or detectable distance (e.g. 30 mm or shorter) from the touch screen.

In the ultrasonic image apparatus 10 according to an embodiment, the driver 12 is connected to each of the plurality of transducers 13 and performs operations for outputting an ultrasonic signal and receiving a feedback signal, and a detection circuit 151 provided in each of a plurality of driving circuits detects whether any of the plurality of transducers 13 is defective. The ultrasonic image apparatus 10 stops the operations of the driving circuit corresponding to at least one defective transducer among the plurality of transducers 13 based on the defect detection results from the detection circuit 151 with regard to the plurality of transducers 13.

According to an embodiment, there is a determination whether any of the plurality of transducers 13 is defective or not before the ultrasonic measurement, thereby ensuring the reliability of the transducer. Further, the defective transducer among the plurality of transducers 13 is inactivated before the ultrasonic measurement to thereby ensure safety.

According to an embodiment, the ultrasonic image apparatus 10 includes the plurality of transducers 13 provided in a form of a matrix-array, but embodiments are not limited thereto. For example, the plurality of transducers 13 may be provided in the form of a one-dimensional or a two-dimensional matrix-array.

The plurality of transducers 13 output an ultrasonic wave for diagnosing an object 19. Specifically, the plurality of transducers 13 are placed in contact with the object 19, output an ultrasonic signal to the object 19, and receive an ultrasonic feedback signal reflected from the object 19.

According to an embodiment, the plurality of transducers 13 may be achieved by using a piezoelectric (PZT) transducer including a plurality of piezoelectric devices, but embodiments are not limited thereto. The plurality of piezoelectric devices may be formed by dividing a piezoelectric material into a plurality of parts. For example, the piezoelectric device may be manufactured by dicing a piezoelectric material, pressing the piezoelectric material against a metal mold, or using various other methods as known to those skilled in the art. The piezoelectric material may include piezoelectric ceramic, single crystal, or composite piezoelectric materials that consist of this material and a polymer, which causes a piezoelectric phenomenon.

The plurality of transducers 13 may have a linear array or a convex array, but embodiments are not limited thereto. Further, the plurality of transducers 13 may have a phased array of duplex-layers, double layers or multi-layers. The array of the plurality of transducers 13 may vary depending on a designer's intention, and a cover may be provided over the arrayed transducers and cover one or more of the plurality of transducers 13.

According to an embodiment, the plurality of transducers 13 may be achieved by using various kinds of ultrasonic transducers such as a piezoelectric ultrasonic transducer that uses a piezoelectric effect of a piezoelectric material, a capacitive micromachined ultrasonic transducer (cMUT) that makes a transformation between ultrasonic waves and an electric signal based on variations in electrostatic capacity, a magnetic micromachined ultrasonic transducer (mMUT) that makes a transformation between ultrasonic waves and an electric signal based on variations in magnetic field, an optical ultrasonic detector that makes a transformation between ultrasonic waves and an electric signal based on variations in optical characteristics, and the like.

If the plurality of transducers 13 is one-dimensionally arrayed on a plane perpendicular to a traveling direction of the ultrasonic waves, it is called a one-dimensional transducer array. The one-dimensional transducer array may include the linear array or the convex array. The one-dimensional transducer array has an advantage of low cost since it is easily manufactured.

If the plurality of transducers 13 is two-dimensionally arrayed on a plane perpendicular to a traveling direction of the ultrasonic waves, it is called a two-dimensional transducer array. The two-dimensional transducer array may also include the linear array or the convex array. The two-dimensional transducer array properly delays input time of signals respectively input thereto, so that the ultrasonic waves can be sent to the object 19 along an external scan line. It is then possible to obtain a three-dimensional image based on a plurality of feedback signals. Therefore, it is easier for the two-dimensional transducer array to make a 3D image.

The plurality of transducers 13 generate an ultrasonic signal in response to a control signal, and emit the generated ultrasonic signal into the object 19. Further, the plurality of transducers 13 receive, i.e. detect, a feedback signal of the ultrasonic waves reflected from certain tissue (e.g. a lesion) inside the object 19. Such reflected ultrasonic waves make the plurality of transducers 13 oscillate, and the plurality of transducers 13 output electrical pulses corresponding to the oscillations. The ultrasonic image apparatus 10 generates an ultrasonic image signal based on the output electrical pulses, and processes the ultrasonic image signal so that an ultrasonic image can be displayed through the display apparatus 200 connected to the ultrasonic image apparatus 10. If anatomical tissues in the object 19 have different ultrasonic wave reflection characteristics, the anatomical tissues are displayed differently, such as with different brightness levels for example, while displaying an ultrasonic image in a brightness (B) mode.

The driver 12 includes a plurality of driving circuits respectively connected to the plurality of transducers 13 for driving the plurality of transducers 13 to output an ultrasonic signal and receive a feedback signal. The driver 12 includes the pulser 121 and the receiver 122 to exchange signals with the plurality of transducers 13. The pulser 121 transmits a pulse to each of the plurality of transducers 13 to output ultrasonic signals, and the receiver 122 receives a feedback signal from each of the plurality of transducers 13 with respect to the pulses.

According to an embodiment, the driver 12 may be achieved by using an analog front-end (AFE) where the pulser 121 and the receiver 122 are combined. Alternatively, the driver 12 may be achieved where the pulser 121 and the receiver 122 are separated. Thus, the driver 12 according to an embodiment may perform the respective operations of the pulser 121 and the receiver 122 regardless of whether the pulser 121 and the receiver 122 are physically separated.

The pulser 121 transmits an electric signal, i.e. a pulse, to the plurality of transducers 13. When the pulser 121 transmits the pulses to the plurality of transducers 13, the plurality of transducers 13 output an ultrasonic wave corresponding to the pulse to the object 19. The transducer 13 then transmits an electric signal, which is converted from the feedback signal of the ultrasonic waves received from the object 19, to the receiver 122. That is, the receiver 122 receives the electric signal of the feedback signal of the ultrasonic waves corresponding to the transmitted pulse from the plurality of transducers 13.

According to an embodiment, the driver 12 includes a multi-channel transceiver to transmit and receive signals through a plurality of channels. For example, the driver 12 may be achieved by using an application specific integrated circuit (ASIC) such as a 64-CH transceiver, a 128-CH transceiver, and the like. The ASIC refers to an integrated circuit customized for a user's specific purpose. According to an embodiment, the ASIC may also serve as an AFE.

According to an embodiment, the pulser 121 and the receiver 122 included in the driver 12 may be achieved by using a single ASIC. Alternatively, the pulser 121 and the receiver 122 may be respectively achieved by using separate ASICs.

The power supply 11 supplies power to the driver 12. The power supply includes a power receiver (not shown) for receiving source power, a rectifier/smoother (not shown) for rectifying and smoothing the received source power, and a power transformer (not shown) for transforming a level of the power output from the rectifier/smoother and supplying it to the driver 12.

According to an embodiment, the power receiver may receive source power from a battery accommodated inside the ultrasonic image apparatus 10. Alternatively, the power receiver may receive source power from an external power supply (not shown). In this case, the power receiver may receive source power from the external power supply through a connector (not shown).

According to an embodiment, the power receiver may further include a power filter for filtering the source power to remove noise.

According to an embodiment, the rectifier/smoother rectifies and smoothes the source power received in and filtered by the power receiver. The rectifier/smoother also rectifies a current output from the power receiver, thereby converting alternating current (AC) into direct current (DC). The rectifier/smoother may include a bridge diode to rectify the current.

The rectifier/smoother may include a smoothing capacitor for smoothing DC voltage output as the current output from the power receiver is rectified. The rectifier/smoother boosts the voltage charged in the smoothing capacitor and thus, improves a power factor of the power supply 11.

According to an embodiment, the power transformer transforms the level of the voltage output from the rectifier/smoother and supplies the power to the driver 12. At this time, the power transformer may also supply power to the pulser 121 and the receiver 122, respectively.

The power transformer may, for example, include a DC-DC converter that transforms the level of the DC voltage output from the rectifier/smoother and outputs a DC voltage having a different level.

The beam-former 16 may perform beam forming so that an ultrasonic signal output through the plurality of transducers 13 can be focused on a focusing point based on at least one of a depth, a size and/or a position of the focusing point. According to an embodiment, the beam-former 16 may further perform pre-processing, e.g. a gain control or the like, to create an ultrasonic image in the image processor 17.

The image processor 17 generates an ultrasonic image signal based on the feedback signal received through the plurality of transducers 13. According to an embodiment, the image processor 17 may support a plurality of modes, and generate an ultrasonic image corresponding to each mode.

The kind of ultrasonic image may be classified into a brightness (B) mode image in which a level of an ultrasonic feedback signal reflected from an object is represented with brightness, a Doppler mode image in which an image of a moving object is represented in the form of spectrums based on Doppler effect, a motion (M) mode image which shows a motion of an object at a certain position over an elapsed time, an elastic mode image which shows an image corresponding to difference in reaction between when an object is pressed and when the object is not pressed, a color (C) mode image in which a speed of a moving object is represented with colors based on Doppler effect, and so forth. Further, the kind of ultrasonic image may be classified into 1D (dimensional), 2D, 3D and 4D mode images, according to display dimensions.

According to an embodiment, the ultrasonic image apparatus 10 may further include a storage (not shown). The storage, which can be a memory provided inside the ultrasonic image apparatus 10, may be achieved by using a nonvolatile storage medium such as a flash-memory. The storage stores ultrasonic image data generated in the image processor 17 and data related to the ultrasonic image. The data to be stored in the storage is not limited to this embodiment, and may include all data related to generation, output and management of the ultrasonic image.

The communicator 18 communicates with the external display apparatus 200. For example, the communicator 18 may use a wireless communication method such as Wi-Fi, Bluetooth, and the like, to communicate with the external display apparatus 200. For example, an ultrasonic image signal generated in the image processor 17 may be transmitted to the external display apparatus 200 through the communicator 18. Thus, an ultrasonic image generated by diagnosis of the object 19 in the ultrasonic image apparatus 10 is displayable through a smart phone, a tablet computer, or the like, as the connected display apparatus.

The detection circuit 151 is provided corresponding to each of the plurality of driving circuits that belong to the driver 12, and detects whether any of the plurality of transducers 13 is defective. The detection circuit 151 may be automatically activated when the ultrasonic image apparatus 10 is turned on or when activated by a user command. The detection circuit 151 detects a defect with regard to the plurality of transducers 13 before the plurality of transducers 13 start the ultrasonic measurement. That is, the detection circuit 151 is automatically activated to detect a defect before the plurality of driving circuits provided in the driver 12 output an ultrasonic signal to each of the plurality of transducers 13 and receive a feedback signal. Thus, the ultrasonic image apparatus 10 according to an embodiment detects whether there is a defective transducer among the plurality of transducers 13 before starting the ultrasonic measurement, thereby taking action with regard to the defective transducer or determining whether to use the apparatus 10.

According to an embodiment, the detection circuit 151 detects whether any of the plurality of transducers 13 is defective or not based on whether a voltage passed through one or more of the plurality of transducers 13 is out of a preset range. For example, the detection circuit 151 may detect whether a voltage drop occurs as compared with a preset voltage while an electric current of a current source passes through each of the plurality of transducers 13. At this time, the detection circuit 151 may determine that a corresponding transducer 13 is defective if the reduced voltage is lower than the preset voltage, and output a logic value of ‘1’. The detection circuit 151 may determine that a corresponding transducer 13 is normal if there are no voltage drops, and output a logic value of ‘0’.

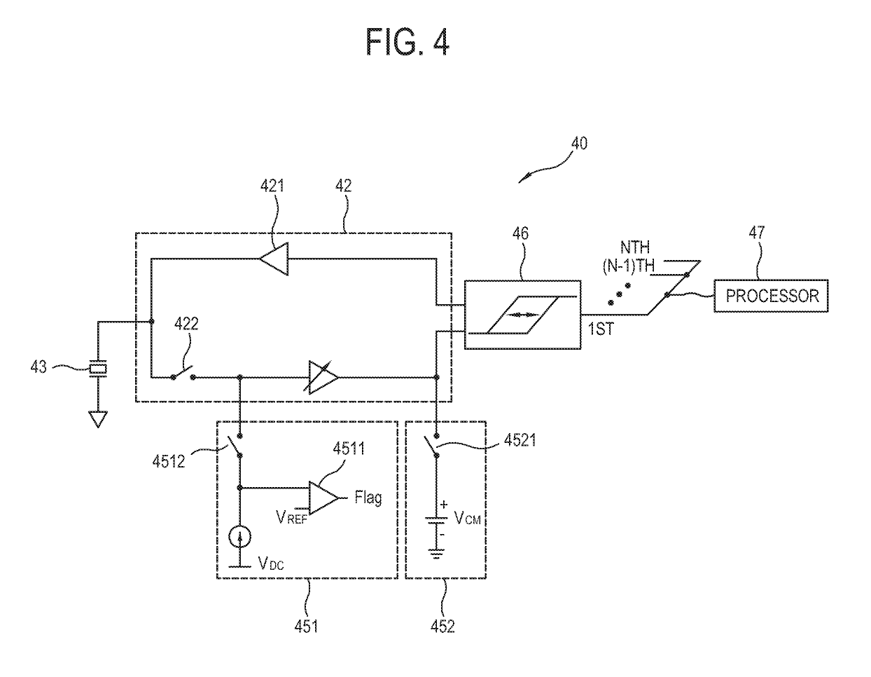

According to an embodiment, the detection circuit 151 may include a low-voltage current source and a comparator, but embodiments are not limited thereto. For example, as shown in FIG. 4, a detection circuit 451 may use a low voltage direct current (VDC) to detect whether an individual transducer 43 is defective.

FIG. 4 illustrates a structure of an application-specific integrated circuit (ASIC) with respect to an individual transducer according to an embodiment of the present disclosure.

Referring to FIG. 4, the detection circuit 451 can more stably detect a defect when using a low voltage instead of using a high voltage. If the transducer 43 is defective, not only is the corresponding transducer 43 influenced by high voltage when the high voltage is applied thereto, but also the neighboring transducers are influenced by the high voltage when the high voltage is applied thereto. However, an applied low voltage can minimize this influence. Further, if a low voltage is used, a small area is sufficient to mount a plurality of devices such as a transistor, a resistor, and so forth that are used for the detection circuit 451, and is thus advantageous when forming the ASIC.

Referring to FIGS. 1 and 4, the detection circuit 151 may include a comparator for comparing voltages respectively passing through one or more of the plurality of transducers 13 with the preset voltage. In FIG. 4, a comparator 4511 may generate a flag value based on results from the comparison with the preset voltage reference (VREF). The comparator 4511 outputs ‘1’ as the flag value of the transducer 43 if the voltage passing through the transducer 43 drops below the preset voltage VREF, and outputs ‘0’ as the flag value if the voltage does not drop below the preset voltage VREF.

According to an embodiment, the voltage setting circuit 152 may be further provided corresponding to each of the plurality of driving circuits of the driver 12, and output a voltage having a preset level corresponding to at least one transducer detected as defective by the detection circuit 151. Further, the voltage setting circuit 152 may include a switch for controlling the output of the voltage having the preset level. In FIG. 4, if the detection circuit 451 detects that the transducer 43 is defective, that is, if the comparator 4511 outputs a flag value of ‘1’, a switch 4521 of a voltage setting circuit 452 is turned on to output a common voltage (VCM) having a preset level. At this time, the voltage VCM having the preset level may be for example set as a common voltage equal to the voltage used in a driving circuit 42.

If the defective transducer makes a large difference between the voltage output from the transducer and the common voltage, image quality is lowered. To solve this problem, the voltage setting circuit 452 controls the defective transducer to output the common voltage.

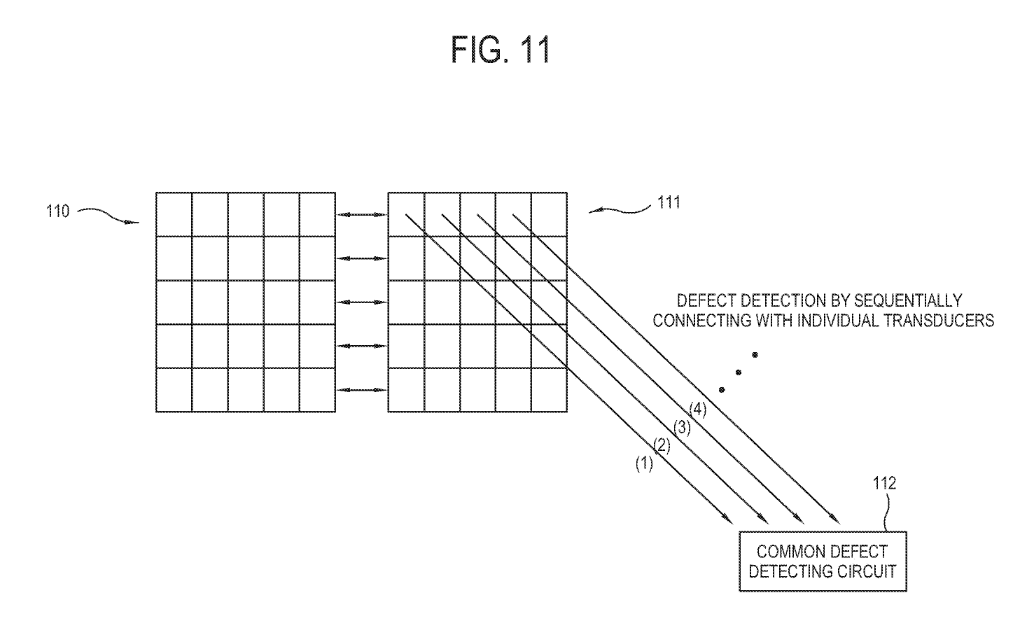

According to an embodiment, the detection circuit 151 is provided as a circuit corresponding to the plurality of driving circuits of the driver 12, and connects the plurality of driving circuits in sequence, thereby detecting whether any of the plurality of transducers 13 is defective. For example, as shown in FIG. 11, to detect whether a plurality of transducers 110 having a two-dimensional matrix-array is defective, a single common defect detecting circuit 112 may be provided instead of individual detection circuits with regard to the plurality of driving circuits 111 corresponding to the plurality of transducers 110.

FIG. 11 illustrates an operation for detecting a defect in each of the plurality of transducers through a common defect detecting circuit according to an embodiment of the present disclosure.

Referring to FIG. 11, the detection circuit provided in each of the plurality of driving circuits 111 is not used to determine whether any of the plurality of transducers 110 is defective. In FIG. 11, a single common defect detecting circuit 112 to which the plurality of driving circuits 111 are sequentially connected is used to determine whether any of the plurality of transducers 110 is defective. The common defect detecting circuit 112 may include a plurality of interface lines (1), (2), (3), (4), and so forth, to respectively connect with the plurality of driving circuits 111, and the plurality of switches for controlling the plurality of driving circuits 111 to be respectively connected in sequence through the interface lines.

According to this embodiment, the detection circuit is not individually provided corresponding to each of the plurality of driving circuits 111, but the single common defect detecting circuit 112 is used to detect whether any of the plurality of transducers 110 is defective. Accordingly, it is possible to make a more simple circuit.

Returning to FIG. 1, the controller 14 controls the driving circuit to stop operations corresponding to at least one defective transducer among the plurality of transducers 13 based on the defect detection results from the detection circuit 12 with respect to each of the plurality of transducers 13. As shown in FIG. 4, if the detection circuit 451 detects that the transducer 43 is defective, that is, if the comparator 4511 outputs the flag value of ‘1’, a driving switch 422 of the driving circuit 42 is turned off to stop the operation of the driving circuit 42. Thus, there is a determination whether the transducer 43 is defective before the ultrasonic measurement, and the driving circuit 42 is inactivated when the transducer 43 is defective, thereby preventing overheating due to a current leakage or the like.

According to an embodiment, the controller 14 stops the operations of the detection circuit 151 corresponding to the defective transducer among the plurality of transducers 13. For example, the controller 14 controls the detection circuit 151 to detect whether any transducer among the plurality of transducers 13 is defective based on whether there is a drop in voltage as compared with the preset voltage with respect to each of the plurality of transducers 13, and inactivates the detection circuit 151 with respect to the corresponding transducer of which the detection is completed. That is, when the ultrasonic image apparatus 10 is booted up, the detection circuit 151 is automatically activated to detect whether the plurality of transducers 13 is defective before the ultrasonic measurement, and is automatically inactivated with regard to the corresponding transducer after each of the plurality of transducers 13 undergoes the detection.

According to an embodiment, the controller 14 controls the supply of power based on a proportion of stopped driving circuits, to the plurality of driving circuits of the driver 12 in accordance with the defect detection results from the detection circuit 151. For example, if the proportion of the transducers of which the driving circuits are stopped as they are detected as defective by the detection circuit 151, to the plurality of transducers 13, is higher than a preset value, the ultrasonic image apparatus 10 may be automatically powered off and prevent performance of the ultrasonic measurement.

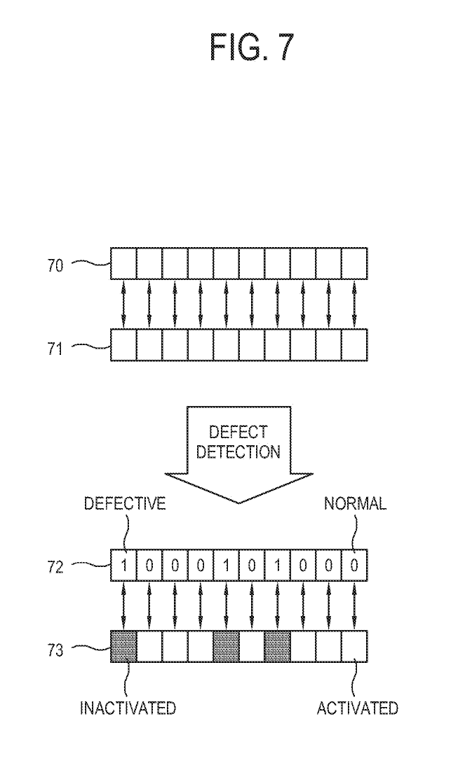

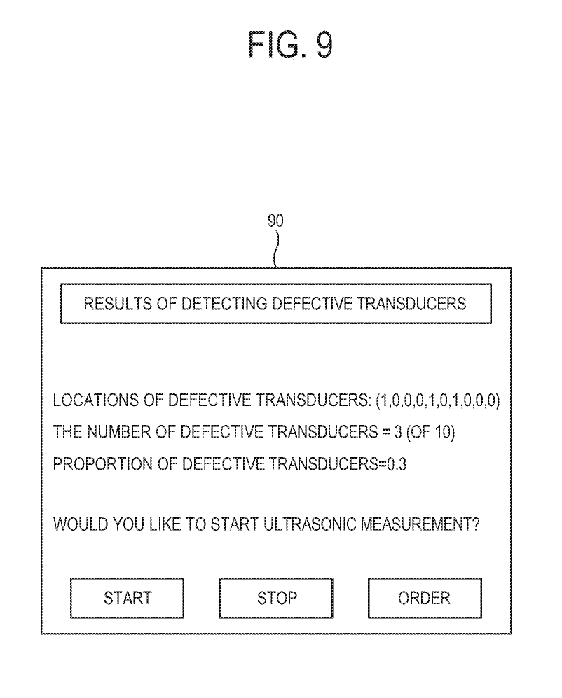

According to an embodiment, the ultrasonic image apparatus 10 may further include a display as shown in FIGS. 7 and 9.

FIG. 7 illustrates an operation for detecting a defect in a one-dimensional matrix-array transducer according to an embodiment of the present disclosure.

FIG. 9 shows results from detecting a defect in a one-dimensional matrix-array transducer according to an embodiment of the present disclosure.

Referring to FIGS. 7 and 9, a display 90 may be achieved in various forms by using a plasma display panel (PDP), a liquid crystal display (LCD), an organic light emitting diode (OLED), a flexible display, and the like. The controller 14 may control the display 90 to display the defect detection results from the detection circuit 151 with respect to each of the plurality of transducers 13. For example, as shown in FIG. 9, the display 90 may display the locations of defective transducers 72, the number of defective transducers, the proportions of defective transducers, and so forth, as the results of the defect detection of the plurality of transducers 72 having the one-dimensional matrix-array. Thus, a user may select whether to start or stop the ultrasonic measurement in accordance with the results of the defect detection of the plurality of transducers 72 displayed on the display 90.

According to an embodiment, the results of the defect detection of the plurality of transducers 72 may also be output as a sound through an audio output unit (not shown). In this case, the locations, the number, the proportion, and so forth, of the defective transducers may be output as a sound or sounds. Further, the results of the defect detection of the plurality of transducers 72 may be represented with certain colors on a screen or a partial area of the apparatus. Alternatively, the display 90 may inform a user when the ultrasonic image apparatus has to be replaced or discarded based on the results of the defect detection of the plurality of transducers 72.

According to an embodiment, the ultrasonic image apparatus 10 may further include the communicator 18 to connect with the external display apparatus 200, and the controller 14 may control the communicator 18 to send the defect detection results from the detection circuit 151 to the display apparatus 200 with respect to each of the plurality of transducers 13. Thus, information about the locations, number and proportion, and the like, of defective transducers of the plurality of transducers 13 is transmitted to the display apparatus 200 connected with the ultrasonic image apparatus 10 so that a user can be informed of the defect detection results.

The method of providing the results of the defect detection of the plurality of transducers 72 is not limited to the foregoing embodiments, but may be variously provided and/or communicated to a user.

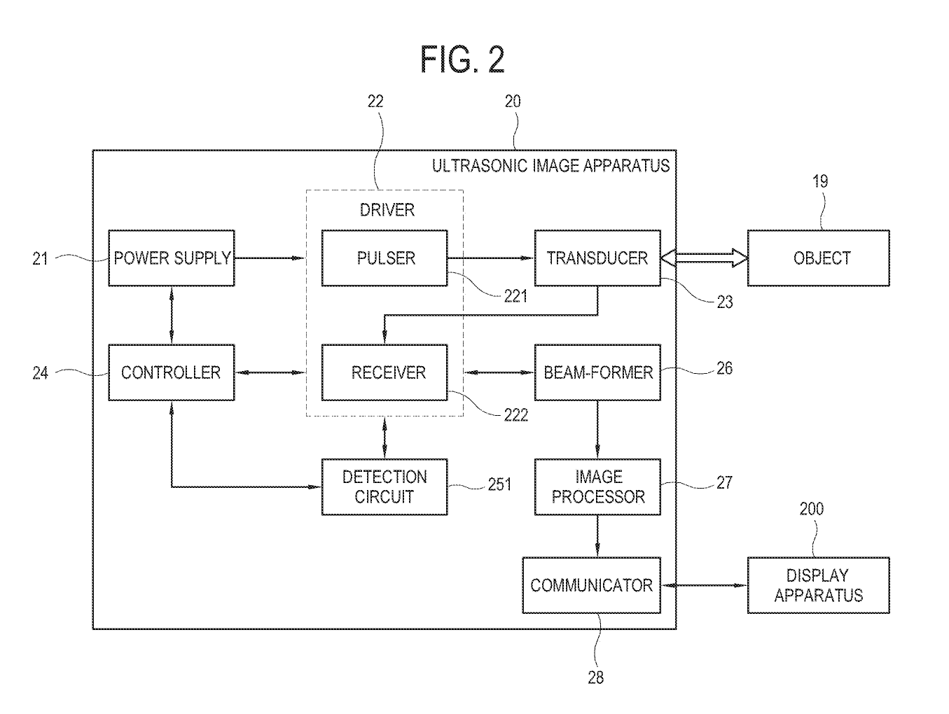

FIG. 2 is a block diagram of an ultrasonic image apparatus according to an embodiment of the present disclosure.

Referring to FIG. 2, an ultrasonic image apparatus 20 according to an embodiment includes a power supply 21, a driver 22, a plurality of transducers 23, a controller 24, a detection circuit 251, a beam-former 26, an image processor 27 and a communicator 28. The driver 22 includes a pulser 221 and a receiver 222. According to an embodiment, the ultrasonic image apparatus 20 may communicate with an external display apparatus 200 through the communicator 28. The object 19, the display apparatus 200, the power supply 21, the driver 22, the transducer 23, the controller 24, the detection circuit 251, the beam-former 26, the image processor 27 and the communicator 28 of the ultrasonic image apparatus 20 shown in FIG. 2 are respectively equivalent to the object 19, the display apparatus 200, the power supply 11, the driver 12, the transducer 13, the controller 14, the detection circuit 151, the beam-former 16, the image processor 17 and the communicator 18 shown in FIG. 1, and thus repetitive descriptions thereof are omitted.

According to an embodiment, the detection circuit 251 is provided in each of a plurality of driving circuits of the driver 22, and detects whether any of the plurality of transducers 23 is defective. The detection circuit 251 detects whether any of the plurality of transducers 23 is defective based on whether a voltage passed through one or more of the plurality of transducers 23 is out of a preset range. For example, the detection circuit 251 may determine that a corresponding transducer is defective if the passed voltage is equal to or lower than the preset voltage, and that the corresponding transducer is normal if the passed voltage is higher than the preset voltage.

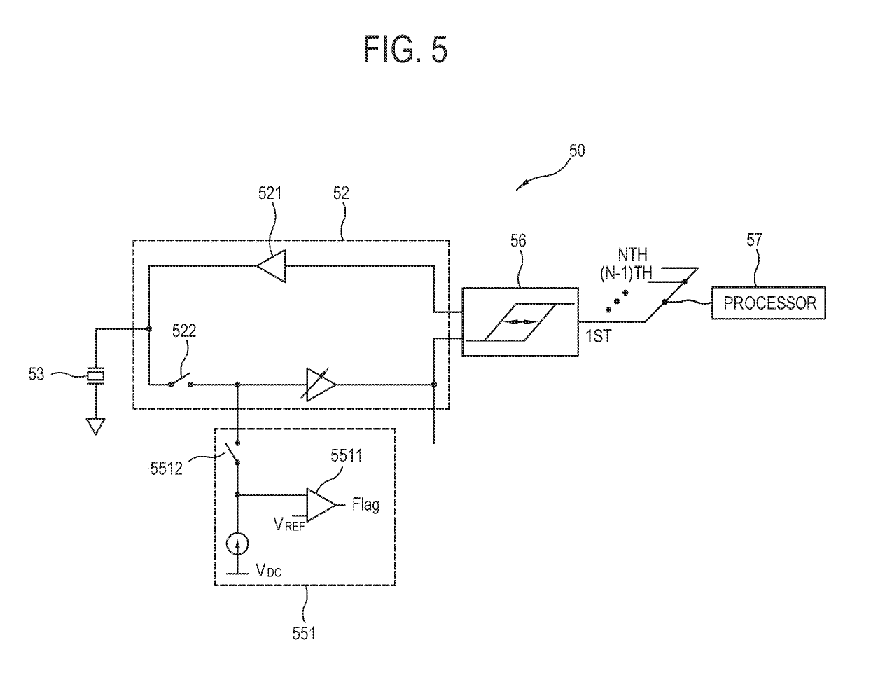

The controller 24 may control the driving circuit to stop operations corresponding to at least one defective transducer among the plurality of transducers 23 based on the defect detection results from the detection circuit 12 with respect to each of the plurality of transducers 23. For example, as shown in FIG. 5, if the detection circuit 551 detects that an individual transducer 53 is defective, that is, if a comparator 5511 outputs the flag value of ‘1’, a driving switch 522 of a driving circuit 52 is turned off to stop the operations of the driving circuit 52.

FIG. 5 illustrates a structure of an ASIC with respect to an individual transducer according to an embodiment of the present disclosure.

Referring to FIGS. 2 and 5, if the detection circuit 551 detects that the transducer 53 is normal, that is, if the comparator 5511 outputs the flag value of ‘0’, the driving switch 522 of the driving circuit 52 is turned on to normally operate the driving circuit 52.

According to this embodiment, the ultrasonic image apparatus 20 includes the detection circuit 251 with respect to each of the plurality of driving circuits to detect whether any of the plurality of transducers 23 is defective, and inactivates the driving circuit 52 before the ultrasonic measurement if the corresponding transducer is defective to thereby ensure safety.

According to an embodiment, the controller 24 may control the driver 22 to output a common voltage, which is equal to a voltage used in the driving circuit, to at least one defective transducer 23 based on the defect detection results from the detection circuit 251 with respect to each of the plurality of transducers 23. Thus, it is possible to prevent image quality from being lowered due to the defective transducer.

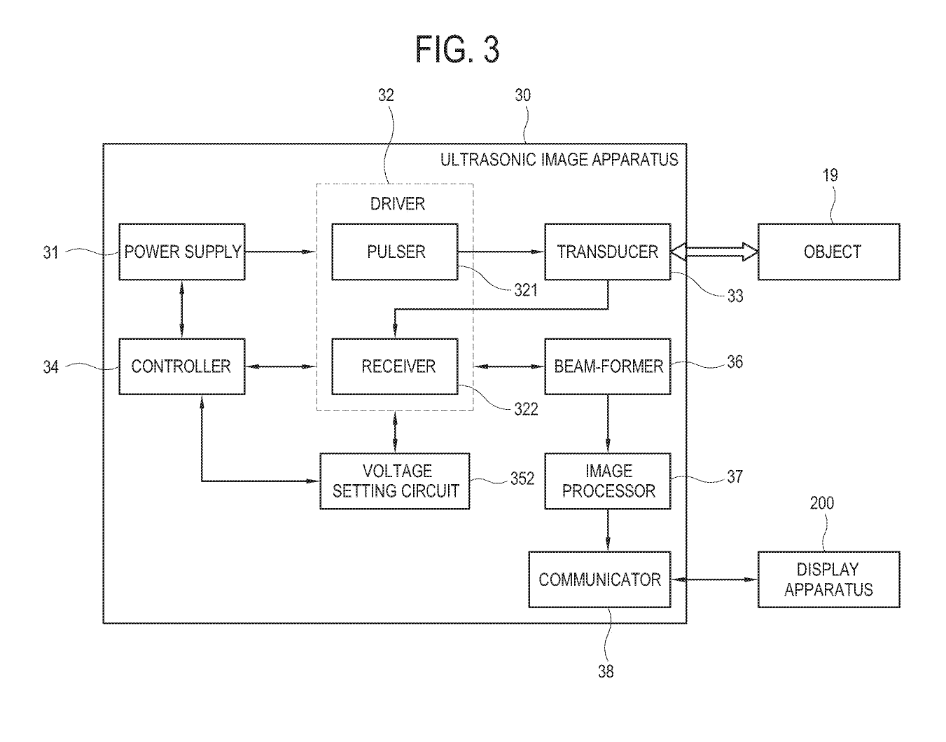

FIG. 3 is a block diagram of an ultrasonic image apparatus according to an embodiment of the present disclosure.

Referring to FIG. 3, an ultrasonic image apparatus 30 according to an embodiment includes a power supply 31, a driver 32, a plurality of transducers 33, a controller 34, a voltage setting circuit 352, a beam-former 36, an image processor 37 and a communicator 38. The driver 32 includes a pulser 321 and a receiver 322. According to an embodiment, the ultrasonic image apparatus 30 may communicate with an external display apparatus 200 through the communicator 38. The object 19, the display apparatus 200, the power supply 31, the driver 32, the transducers 33, the controller 34, the voltage setting circuit 352, the beam-former 36, the image processor 37 and the communicator 38 of the ultrasonic image apparatus 30 shown in FIG. 3 are equivalent to the object 19, the display apparatus 200, the power supply 11, the driver 12, the transducer 13, the controller 14, the voltage setting circuit 152, the beam-former 16, the image processor 17 and the communicator 18 of FIG. 1, and thus repetitive descriptions thereof are omitted.

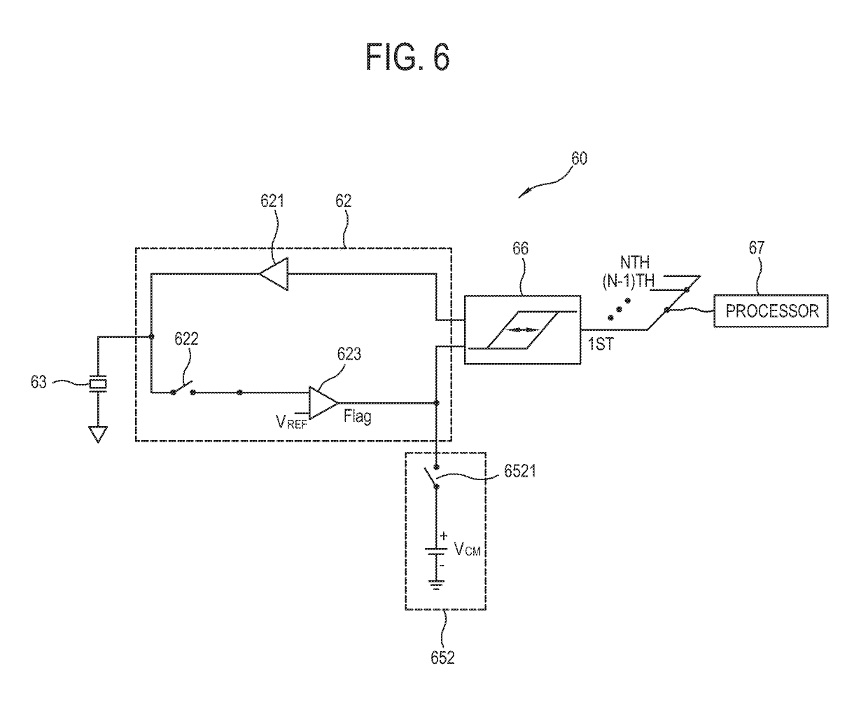

The driver 32 may use the plurality of driving circuits itself to detect whether any of the plurality of transducers 33 is defective. That is, without a separate detection circuit, the driving circuit itself may perform the function of the detection circuit. In this case, the plurality of driving circuits may include a comparator for detecting whether any of the plurality of transducers 33 is defective. For example, as shown in FIG. 6, a driving circuit 62 includes a comparator 623, and the comparator 623 outputs a flag value of ‘1’ if a voltage passed through a transducer 63 is equal to or lower than a preset voltage VREF of the comparator 623 and outputs a flag value of ‘0’ if the passed voltage is higher than the preset voltage VREF.

FIG. 6 illustrates a structure of an ASIC with respect to an individual transducer according to an embodiment of the present disclosure.

Referring to FIGS. 3 and 6, the driving circuit 62 detects that the transducer 63 is defective if the comparator 623 outputs the flag value of ‘1’, and detects that the transducer 63 is normal if the output flag value is ‘0’.

The controller 34 may control the driving circuit to stop operations corresponding to at least one defective transducer among the plurality of transducers 33 based on the defect detection results from the driving circuits itself with respect to the plurality of transducers 33. For example, in FIG. 6, if the driving circuit 62 detects that the transducer 63 is defective, that is, if the comparator 623 outputs a flag value of ‘1’, a driving switch 622 of the driving circuit 62 is turned off to stop the operations of the driving circuit 62.

The voltage setting circuit 352 is provided corresponding to each of the plurality of driving circuits of the driver 32, and outputs a voltage having a preset level corresponding to at least one transducer detected as defective. For example, in FIG. 6, if the driving circuit 62 detects that the transducer 63 is defective, the voltage setting circuit 652 turns on a switch 6521 to output the common voltage VCM equal to a voltage used in the driving circuit 62 to the defective transducer 63. Thus, it is possible to create an ultrasonic image without lowering image quality even though at least one transducer is defective.

Referring again to FIG. 4, an ultrasonic image apparatus 40 includes the 1^{st}, 2^{nd}, . . . , (N−1)^{th} and N^{th} transducers; the 1^{st}, 2^{nd}, . . . , (N−1)^{th} and N^{th} driving circuits and beam-formers 46 corresponding to the transducers; and a processor 47 for processing data received therefrom. Here, N driving circuits corresponding to N transducers are respectively achieved by using ASICs, and each driving circuit includes a pulser and a receiver.

According to an embodiment, the detection circuit 451 and a voltage setting circuit 452 may be provided corresponding to the driving circuit 42 for driving the transducer 43 among the N transducers.

The detection circuit 451 may use a low voltage VDC to detect whether the transducer 43 is defective. The detection circuit 451 can more stably detect a defect when using a low voltage instead of using a high voltage. If the transducer 43 is defective, not only is the corresponding transducer 43 influenced by high voltage when the high voltage is applied thereto, but also the neighboring transducers are influenced by the high voltage when the high voltage is applied thereto. However, an applied low voltage can minimize this influence. Further, if a low voltage is used, a small area is sufficient to mount a plurality of devices such as a transistor, a resistor, and so forth that are used for the detection circuit 451, and is thus advantageous to form the ASIC.

The detection circuit 451 may include the comparator 4511 for comparing a voltage passing through the transducer 43 with the preset voltage VREF. The comparator 4511 may generate a flag based on the results of the comparison with the preset voltage VREF. The comparator 4511 outputs ‘1’ as the flag value of the transducer 43 if the voltage passing through the transducer 43 drops below the preset voltage VREF, and outputs ‘0’ as the flag value if the voltage does not drop below the preset voltage VREF.

The voltage setting circuit 452 is provided with respect to the driving circuit 42, and outputs a voltage VCM having a preset level if the transducer 43 is detected as defective by the detection circuit 451. The voltage setting circuit 452 may include a switch 4521 for controlling the output of the voltage VCM having the preset level. For example, if the detection circuit 451 detects that the transducer 43 is defective, that is, if the comparator 4511 outputs a flag value of ‘1’, the switch 4521 of the voltage setting circuit 452 is turned on to output a voltage VCM having a preset level. The voltage VCM having the preset level may be for example set as a common voltage equal to the voltage used in the driving circuit 42.

According to an embodiment, if the detection circuit 451 detects that the transducer 43 is defective, the driving switch 422 of the driving circuit 42 is turned off to thereby stop the operations of the driving circuit 42 by for example, opening a circuit including element 421. Thus, there is a determination whether the transducer 43 is defective before the ultrasonic measurement, and the driving circuit 42 is inactivated when the transducer 43 is defective thereby preventing overheating due to a current leakage or the like.

The detection circuit 451 may further include a detection switch 4512 for controlling whether to perform the detection. For example, if the ultrasonic image apparatus 40 is booted up, the detection circuit 451 turns on the detection switch 4512 to perform the defect detection of the transducer 43 before the ultrasonic measurement, and turns off the detection switch 4512 to be inactivated when the defect detection of the transducer 43 is completed. Thus, the detection circuit 451 can be automatically activated or inactivated in accordance with whether the ultrasonic image apparatus 40 is booted up or whether the defect detection is completed.

According to an embodiment, the defect detection results of the detection circuit 451 may be transmitted to the processor 47. That is, the detection circuit 451 sends the processor 47 the results of detecting whether the transducer 43 is defective. For example, the flag value of ‘1’ or ‘0’ output from the comparator 4511 may be transmitted as the defect detection result. The plurality of detection circuits corresponding to the plurality of transducers each transmit defect detection results to the processor 47. Thus, the processor 47 can determine the locations of defective transducers among the plurality of transducers, the number and proportion of defective transducers, and so forth.

Referring again to FIG. 5, an ultrasonic image apparatus 50 includes the 1^{st}, 2^{nd}, . . . , (N−1)^{th} and N^{th} transducers; the 1^{st}, 2^{nd}, . . . , (N−1)^{th} and N^{th} driving circuits and beam-formers 56 corresponding to the transducers; and a processor 57 for processing data received therefrom. Here, N driving circuits corresponding to N transducers are respectively achieved by using ASICs, and each driving circuit includes a pulser and a receiver.

According to an embodiment, the detection circuit 551 may be provided corresponding to the driving circuit 52 for driving the transducer 53 among the N transducers.

A detection circuit 551 may include the comparator 5511 for comparing a voltage passing through the transducer 53 with the preset voltage VREF. The comparator 5511 may generate a flag based on results of the comparison with the preset voltage VREF. The comparator 5511 outputs ‘1’ as the flag value of the transducer 53 if the voltage passing through the transducer 53 drops below the preset voltage VREF, and outputs ‘0’ as the flag value if the voltage does not drop below the preset voltage VREF. The flag value of ‘1’ indicates that the transducer 53 is defective, and the flag value of ‘0’ indicates that the transducer 53 is normal.

The detection circuit 551 may use a low voltage VDC to detect whether the transducer 53 is defective. The detection circuit 551 can more stably detect a defect when using a low voltage VDC instead of using a high voltage.

If the detection circuit 551 detects that the transducer 53 is defective, the driving switch 522 of the driving circuit 52 is turned off to thereby stop the operations of the driving circuit 52 by for example, opening a circuit including element 521. If the detection circuit 551 detects that the transducer 53 is normal, the driving switch 522 of the driving circuit 52 is turned on to thereby make the driving circuit 52 operate normally.

The detection circuit 551 may further include a detection switch 5512 for controlling whether to perform the detection. For example, if the ultrasonic image apparatus 50 is booted up, the detection circuit 551 turns on the detection switch 5512 to perform the defect detection of the transducer 53 before the ultrasonic measurement, and turns off the detection switch 5512 to be inactivated when the defect detection of the transducer 53 is completed. Thus, the detection circuit 551 can be automatically activated or inactivated in accordance with whether the ultrasonic image apparatus 50 is booted up or whether the defect detection is completed.

According to an embodiment, if the detection circuit 551 detects that the transducer 53 is defective, the driving switch 522 of the driving circuit 52 is turned off to stop the operations of the driving circuit 52.

According to an embodiment, the defect detection results from the detection circuit 551 may be sent to the processor 57. The plurality of detection circuits corresponding to the plurality of transducers transmit the respective defect detection results to the processor 57, and thus the processor 57 determines the location of the defective transducer among the plurality of transducers, the number and proportion of defective transducers, and so forth.

Referring again to FIG. 6, an ultrasonic image apparatus 60 includes the 1^{st}, 2^{nd}, . . . , (N−1)^{th} and N^{th} transducers; the 1^{st}, 2^{nd}, . . . , (N−1)^{th} and N^{th} driving circuits and beam-formers 66 corresponding to the transducers; and a processor 67 for processing data received therefrom. Here, N driving circuits corresponding to N transducers are respectively achieved by using ASICs, and each driving circuit includes a pulser and a receiver.

According to an embodiment, a voltage setting circuit 652 may be provided corresponding to the driving circuit 62 for driving the transducer 63 among the N transducers.

According to an embodiment, the driving circuit 62 itself may detect whether the transducer 63 is defective, without a separate detection circuit. The driving circuit 62 includes the comparator 623, and the comparator 623 outputs a flag value of ‘1’ if a voltage passing through the transducer 63 is equal to or lower than a voltage VREF previously set in the comparator 623, and outputs a flag value of ‘0’ if the voltage is higher than the preset voltage VREF. Thus, the driving circuit 62 detects that the transducer 63 is defective if the comparator 623 outputs the flag value of ‘1’, and detects that the transducer 63 is normal if the comparator 623 outputs the flag value of ‘0’.

According to an embodiment, if the driving circuit 62 detects that the transducer 63 is defective, the driving switch 622 of the driving circuit 62 is turned off to stop the operations of the driving circuit 62 by for example, opening a circuit including element 621.

The voltage setting circuit 652 outputs a voltage having a preset level corresponding to the transducer 63 if the driving circuit 62 detects that the transducer 63 is defective. For example, if there is a determination that the transducer 63 is defective, the voltage setting circuit 652 turns on the switch 6521 so that the common voltage VCM equal to the voltage used in the driving circuit 62 can be output to the defective transducer 63. Thus, it is possible to create an ultrasonic image without lowering image quality even though at least one transducer is defective.

Referring again to FIG. 7, a plurality of transducers 70 has a one-dimensional matrix-array, and is connected to each of the plurality of ASICs 71 operating to drive the plurality of transducers 70.

Each of the plurality of ASICs 71 includes a pulser for transmitting a pulse to each of the plurality of transducers 70 to output an ultrasonic signal, and a receiver for receiving a feedback signal corresponding to the transmitted pulse from each of the plurality of transducers 70. The pulser and the receiver may be provided as a single ASIC, or provided as separate ASICs.

According to an embodiment, each of the plurality of ASICs 71 may include a plurality of detection circuits to detect whether any of the plurality of transducers 70 is defective. Further, each of the plurality of ASICs 71 may be provided with a voltage setting circuit to output a common voltage to a defective transducer among the plurality of transducers 70.

Each of the plurality of detection circuits includes a comparator for comparing a voltage passing through each of the plurality of transducers 70 with a preset voltage to detect whether any of the plurality of transducers 70 is defective, and uses a low voltage. The comparator outputs a flag value of ‘1’ if the passed voltage is equal to or lower than the preset voltage, and outputs a flag value of ‘0’ if the passed voltage is higher than the preset voltage. Thus, the value of ‘1’ or ‘0’ is output as a defect detection result with respect to each of the plurality of transducers 72, and transmitted to a system so as to determine the locations, number, proportion, and so forth, of defective transducers.

According to an embodiment, each of the plurality of ASICs 73 can be automatically activated or inactivated in accordance with the results of the defect detection of each of the plurality of transducers 72. For example, a corresponding ASIC is inactivated with respect to a transducer of which the defect detection result is ‘1’, among the plurality of transducers 72, and is activated with respect to a transducer of which the defect detection result is ‘0’. Thus, there is a determination whether the transducer is defective before the ultrasonic measurement, thereby preventing overheating or similar problems associated with the defective transducer.

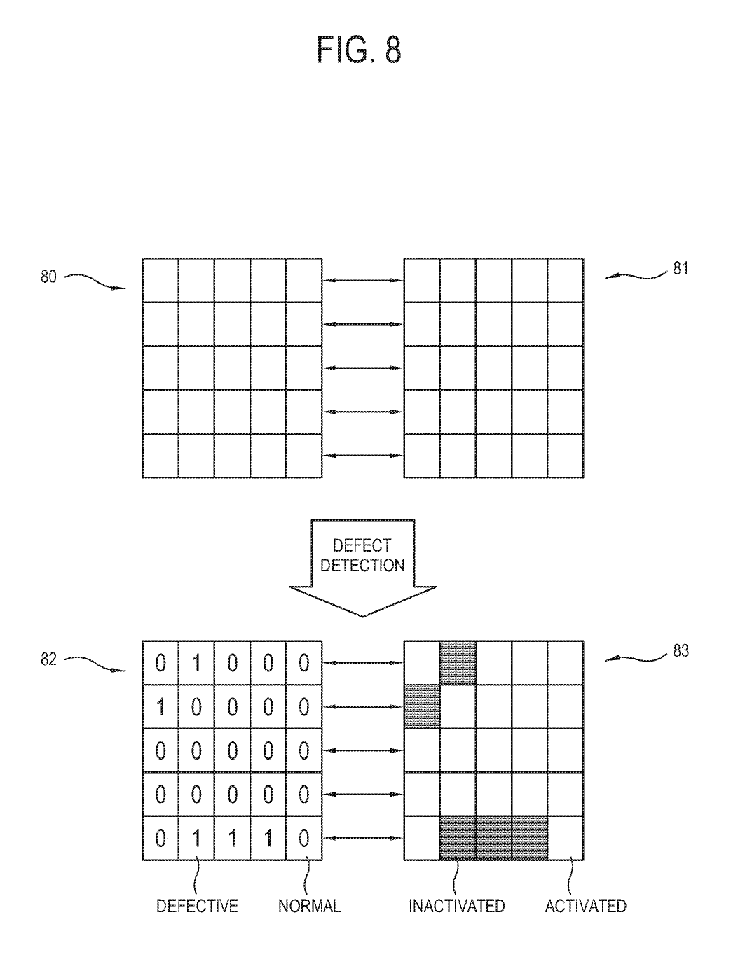

FIG. 8 illustrates an operation of detecting a defect in a two-dimensional matrix-array transducer according to an embodiment of the present disclosure.

Referring to FIG. 8, a plurality of transducers 80 has a two-dimensional matrix-array, and is connected to each of a plurality of ASICs 81 operating to drive the plurality of transducers 80.

Each of the plurality of ASICs 81 includes a pulser for transmitting a pulse to each of the plurality of transducers 80 to output an ultrasonic signal, and a receiver for receiving a feedback signal corresponding to the transmitted pulse from each of the plurality of transducers 80.

According to an embodiment, each of the plurality of ASICs 81 may include a plurality of detection circuits to detect whether any of the plurality of transducers 80 is defective. Further, each of the plurality of ASICs 81 may be provided with a voltage setting circuit to output a common voltage to a defective transducer among the plurality of transducers 80.

Each of the plurality of detection circuits includes a comparator for comparing a voltage passing through each of the plurality of transducers 80 with a preset voltage to detect whether any of the plurality of transducers 80 is defective, and uses a low voltage. The comparator outputs a flag value of ‘1’ if the passed voltage is equal to or lower than the preset voltage, and outputs a flag value of ‘0’ if the passed voltage is higher than the preset voltage. Thus, the value of ‘1’ or ‘0’ is output as a defect detection result with respect to each of the plurality of transducers 82, and transmitted to a system so as to determine the locations, number, proportion, and so forth, of defective transducers.

According to an embodiment, each of a plurality of ASICs 83 can be automatically activated or inactivated in accordance with the results of the defect detection of each of the plurality of transducers 82. For example, a corresponding ASIC is inactivated with respect to the transducer of which the defect detection result is 1′, among the plurality of transducers 82, and is activated with respect to the transducer of which the defect detection result is ‘0’.

Referring again to FIG. 9, the display 90 may display the locations, number, proportion, and so forth, of the defective transducers as the results of the defect detection of the plurality of transducers 72 having the one-dimensional matrix-array of FIG. 7. Thus, a user can select whether to start or stop the ultrasonic measurement in accordance with the results of detected defects in the plurality of transducers 72, displayed on the display 90. According to an embodiment, the display 90 may be included in the external display apparatus 200 connected with the ultrasonic image apparatus. In this case, the results of the detection of defects in the plurality of transducers 72 are sent to the external display apparatus and then displayed on the display 90.

According to an embodiment of the present disclosure, a user determines the number and locations of defective transducers based on the results of the detected defects in the plurality of transducers 72 having the one-dimensional matrix-array displayed on the display 90, and may make a purchase of a transducer by clicking an ‘order’ button if there is a need for replacing the defective transducer. If the ‘order’ button is clicked on the display 90, a web browser may be executed to be automatically linked to a store site for making the purchase of the transducer. Alternatively, if the results of the detected defects in the plurality of transducers 72 are displayed on a screen of a user's smartphone and a user clicks an ‘order’ button, a predetermined application for making a purchase of the transducer may be automatically executed so that the user can easily make an order for the transducer.

According to this embodiment, if a plurality of transducers is defective, it is convenient to view and check the locations and number of the defective transducers through a screen of a user terminal. As necessary, a user may make a purchase of a transducer at the same time when informed that the transducer is defective.

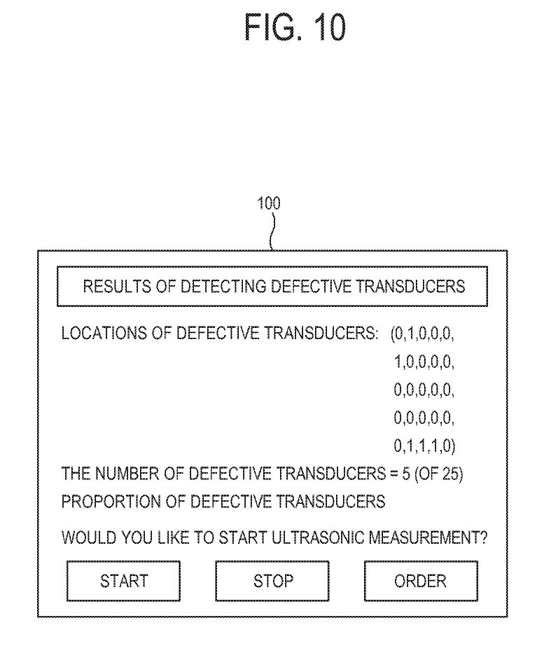

FIG. 10 shows an example of displaying results from detecting a defect in a two-dimensional matrix-array transducer according to an embodiment of the present disclosure.

Referring to FIG. 10, a display 100 may display the locations, number, proportion, and so forth, of defective transducers as the results of the defect detection of the plurality of transducers 82 having the two-dimensional matrix-array of FIG. 8. Thus, a user can select whether to start or stop the ultrasonic measurement in accordance with the results of detected defects in the plurality of transducers 82 displayed on the display 100. According to an embodiment, the display 100 may be included in the external display apparatus 200 connected with the ultrasonic image apparatus. In this case, the results of detecting the defect in the plurality of transducers 82 are sent to the external display apparatus and then displayed on the display 100.

According to an embodiment, a user determines the number and locations of defective transducers based on the results of the detected defects in the plurality of transducers 82 having the two-dimensional matrix-array displayed on the display 100, and may make a purchase of a transducer by clicking an ‘order’ button if there is a need for replacing the defective transducer. If the ‘order’ button is clicked on the display 100, a web browser may be executed to be automatically linked to a store site for making the purchase of the transducer. Alternatively, if the results of the detected defects in the plurality of transducers 82 are displayed on a screen of a user's smartphone and a user clicks an ‘order’ button, a predetermined application for making a purchase of the transducer may be automatically executed so that the user can easily make an order for the transducer.

Referring again to FIG. 11, when it is to be determined whether any of the plurality of transducers 110 having the two-dimensional matrix-array is defective, a single common defect detecting circuit 112 may be used instead of an individual detection circuit with respect to each of the plurality of driving circuits 111 corresponding to the plurality of transducers 110. In this case, the individual detection unit provided in each of the plurality of driving circuits 111 is not used to determine whether any of the plurality of transducers 110 is defective. In FIG. 11, the plurality of driving circuits 111 are sequentially connected to the single common defect detecting circuit 112, thereby detecting whether any of the plurality of transducers 110 is defective. The common defect detecting circuit 112 may include a plurality of interface lines for respectively connecting with the plurality of driving circuits 111, and a plurality of switches for controlling sequential connection of the plurality of driving circuits 111 through the respective interface lines.

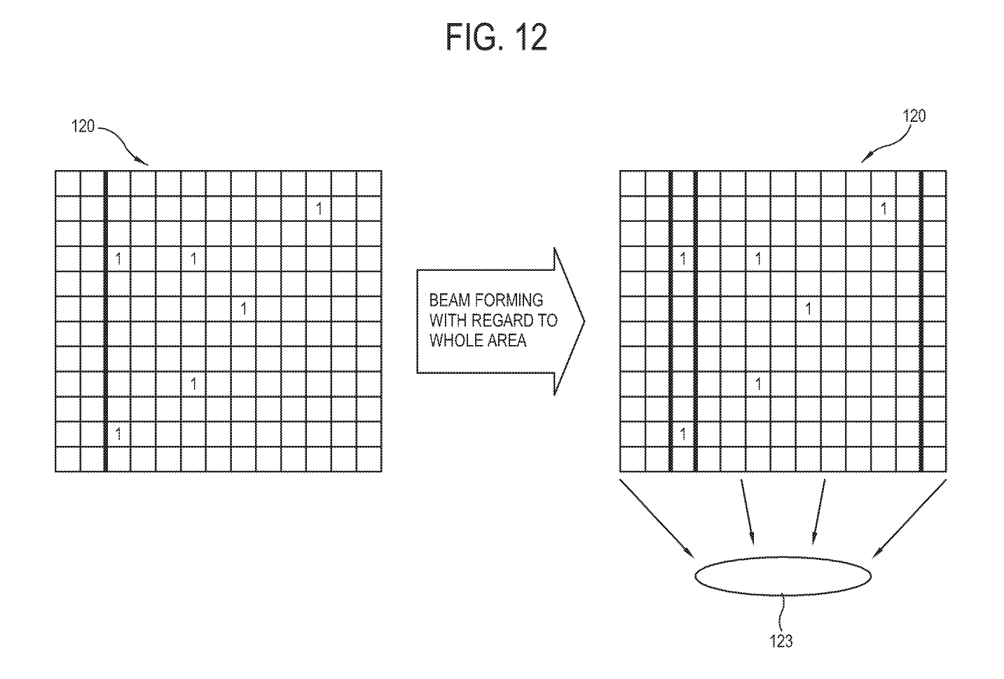

FIG. 12 illustrates a case where defective transducers are widely dispersed according to an embodiment of the present disclosure.

Referring to FIG. 12, based on the results of a defect detection in a plurality of transducers 120, the beam forming may be performed to focus ultrasonic signals output from the plurality of transducers 120 toward an object 123. For example, if the defective transducers corresponding to a value of ‘1’ are widely dispersed throughout the whole area as results of a defect detection in the plurality of transducers 120 having the two-dimensional matrix-array, the beam forming may be applied to the whole area including at least one defective transducer. That is, if the defective transducers are widely dispersed, it has a little influence on the quality of the ultrasonic image and thus the beam forming is applied to the whole area to thereby generate an ultrasonic image.

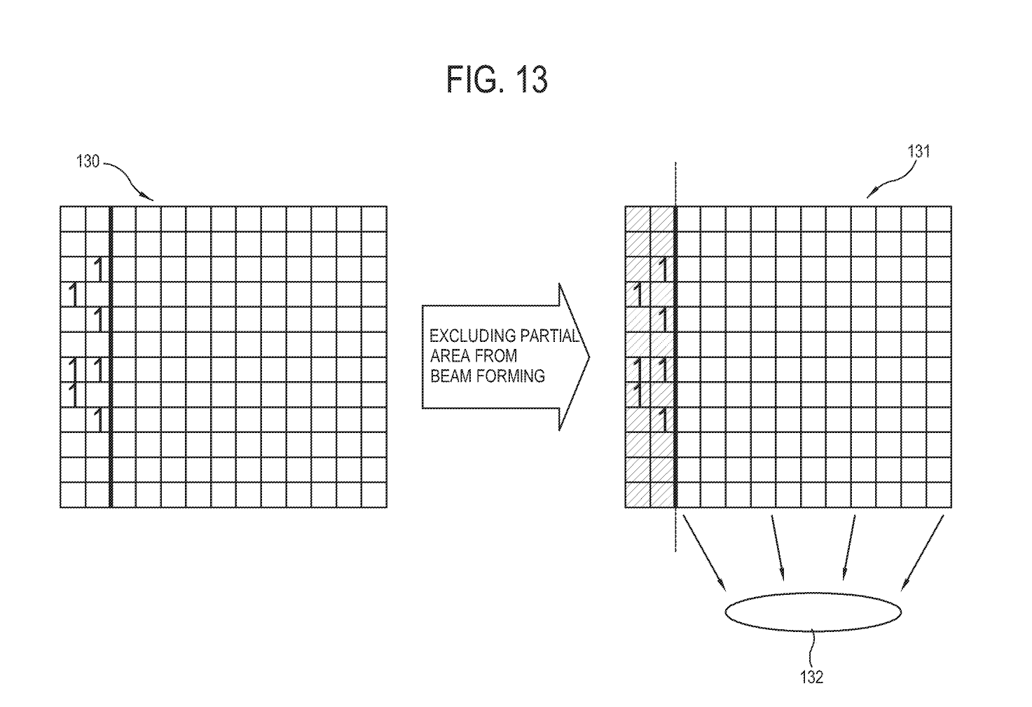

FIG. 13 illustrates a case where defective transducers are densely concentrated in a certain area according to an embodiment of the present disclosure.

Referring to FIG. 13, based on the results of a defect detection in a plurality of transducers 130, the beam forming may be performed so that ultrasonic signals output from the plurality of transducers 131 except those of a certain area can be focused toward an object 132. For example, if the defective transducers corresponding to a value of ‘1’ are densely concentrated at a left area as results of a defect detection in the plurality of transducers 130 having the two-dimensional matrix-array, the beam forming may be applied to the area including the plurality of transducers except for the left area where the defective transducers are densely concentrated. That is, if the defective transducers are densely concentrated in a certain area, it may have a greater influence on the quality of the ultrasonic image and thus, the beam forming is applied to the area except for the area where the defective transducers are densely concentrated, thereby generating an ultrasonic image.

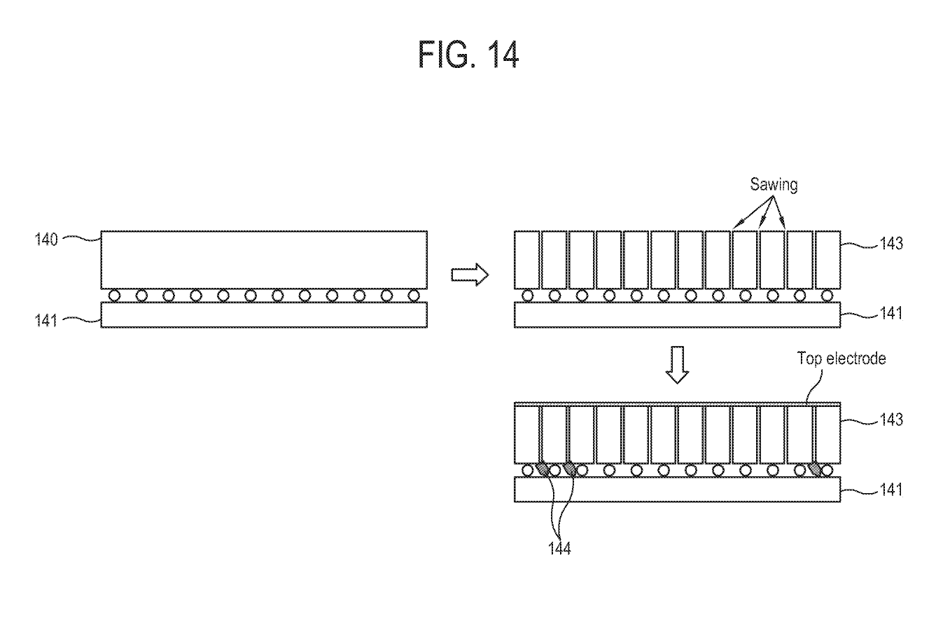

FIG. 14 illustrates a defective PZT transducer according to an embodiment of the present disclosure.

Referring to FIG. 14, a plurality of PZT transducers 143 may be provided by dividing a piezoelectric material 140 to form a plurality of piezoelectric devices. For example, the plurality of piezoelectric devices may be manufactured by dicing the longitudinally formed piezoelectric material 140, pressing the piezoelectric material 140 against a metal mold, or using various other methods as known to those skilled in the art. The piezoelectric material may include piezoelectric ceramic, single crystal, or composite piezoelectric materials that consist of this material and a polymer, which causes a piezoelectric phenomenon.

As illustrated in FIG. 14, any of the plurality of PZT transducers 143 may be defective due to short-circuits caused by metal and the piezoelectric residual material 144 while forming the plurality of piezoelectric devices. Further, any of the plurality of PZT transducers 143 may be defective due to short-circuits caused by physical damage and electric shock. Still further, any of the plurality of PZT transducers 143 may be defective due to depolarization caused by repetitive applications of a high-voltage signal and heat.

Thus, the plurality of PZT transducers 143 may be individually or collectively defective due to various causes. According to an embodiment, the detection circuits are respectively provided with regard to the ASICs 141 for driving the plurality of PZT transducers 143, and it is thus possible to detect whether any of the plurality of PZT transducers 143 is defective. Further, the ASICs are inactivated corresponding to the defective transducers among the plurality of PZT transducers 143, thereby ensuring safety.

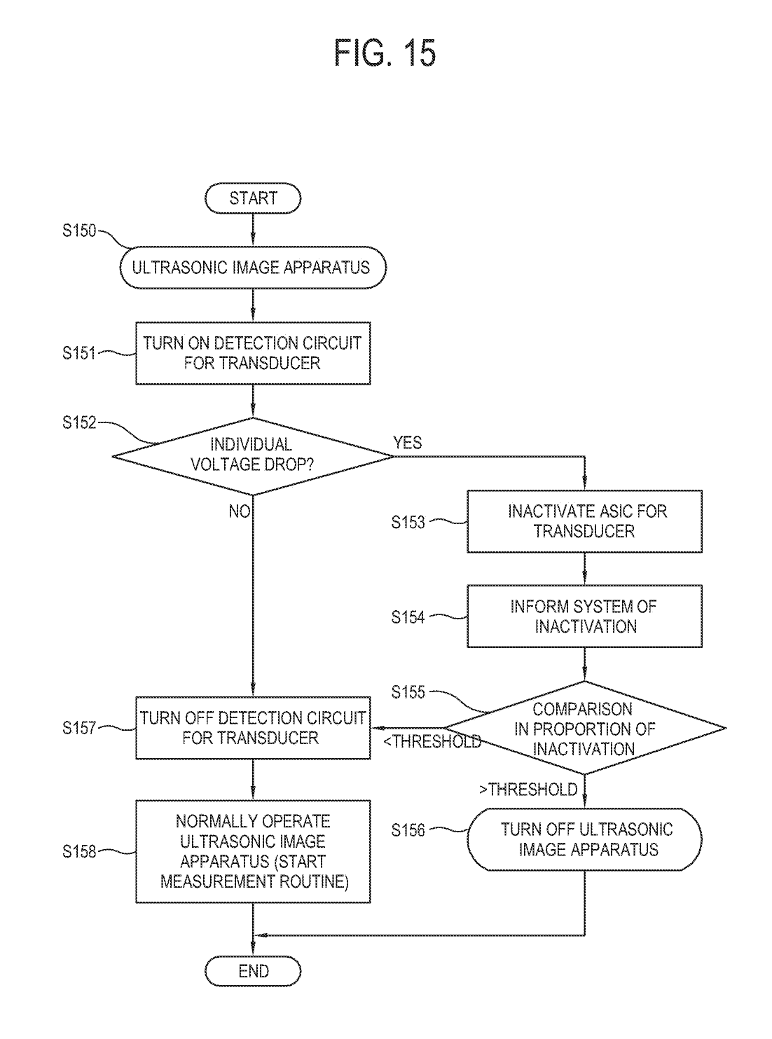

FIG. 15 is a flowchart of a process of protecting a plurality of transducers according to an embodiment of the present disclosure.

Referring to FIG. 15, at operation S150 the ultrasonic image apparatus is first turned on, and at operation S151 a circuit for detecting a defect in any of the plurality of transducers is turned on. Next, at operation S152 it is measured whether there is a voltage drop in each individual transducer.

As a result of the operation S152, if there is a voltage drop in any individual transducer, at operation S153 the ASIC for the corresponding transducer is inactivated. Next, at operation S154 a system is informed that the ASIC for the corresponding transducer is inactivated. At operation S155, a proportion of inactivated ASICs is compared with a predetermined threshold. As a result of the operation S155, if the proportion of inactivated ASICs is equal to or higher than the predetermined threshold, at operation S156 the ultrasonic image apparatus is powered off. If the proportion of inactivated ASICs is lower than the predetermined threshold, at operation S157 the detection circuit for the corresponding transducer is turned off, and at operation S158 the ultrasonic image apparatus performs normal operations.

As a result of the operation S152, if there is no voltage drop in each individual transducer, at operation S157 the detection circuit for each transducer is turned off, and at operation S158 an ultrasonic measurement routine starts to make the ultrasonic image apparatus perform the normal operations.

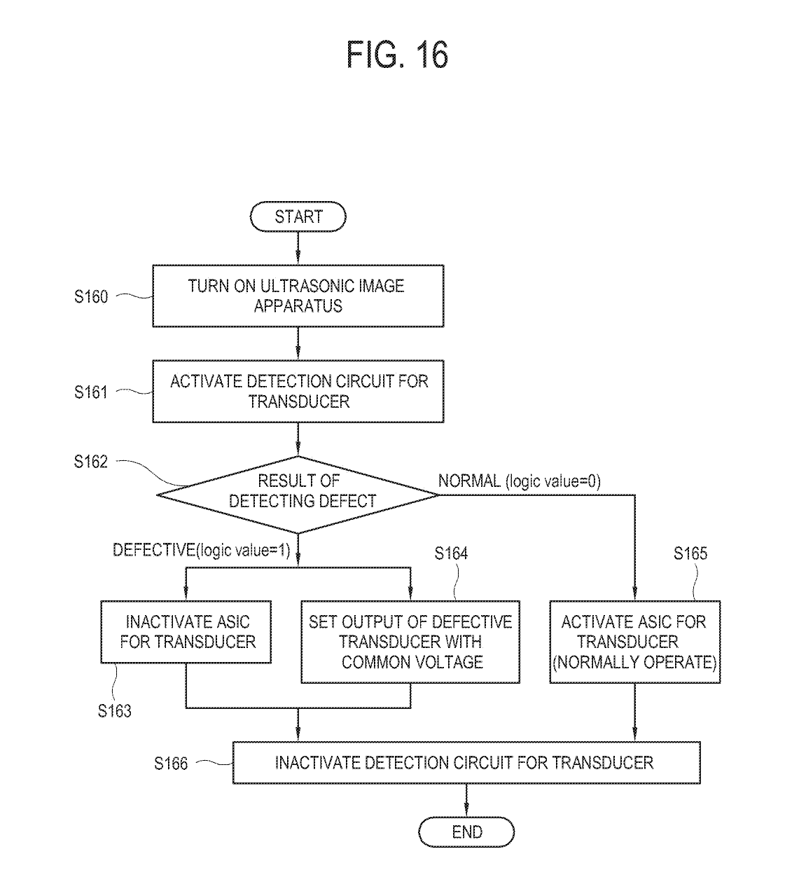

FIG. 16 is a flowchart of a defect detecting process with regard to an individual transducer according to an embodiment of the present disclosure.

Referring to FIG. 16, at operation S160 the ultrasonic image apparatus is powered on, and then at operation S161 the detection circuit for the transducer is activated. At operation S162 it is determined whether the transducer is normal or defective. If the transducer is defective, a logic value of ‘1’ is output. If the transducer is normal, a logic value of ‘0’ is output. As a result of the operation S162, if the transducer is defective, at operation S163 the ASIC corresponding to the defective transducer is inactivated. At the same time, at operation S164 the output of the defective transducer is set with the common voltage. Thus, it is possible to solve overheating or similar safety problems due to the defective transducer, and prevent the quality of the ultrasonic image from being deteriorated due to the defective transducer.

As a result of the operation S162, if the transducer is normal, at operation S165 the ASIC for the normal transducer is activated to perform the normal operations.

At operation S166 the detection circuit for the transducer is inactivated to stop detecting the defect.

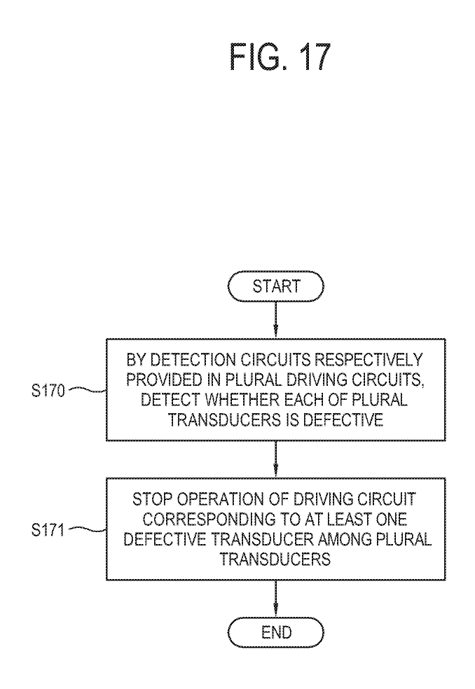

FIG. 17 is a flowchart of a method of controlling an ultrasonic image apparatus according to an embodiment of the present disclosure.

Referring to FIG. 17, at operation S170 it is determined by the detection circuits respectively provided in the plurality of driving circuits whether any of the plurality of transducers is defective. Each of the detection circuits may include a low-voltage current source and a comparator. Operation S170 may include an operation of detecting whether any of the plurality of transducers is defective based on whether a voltage passed through one or more of the plurality of transducers is out of a preset range. According to an embodiment, the operation S170 may further include an operation for stopping the detection circuit corresponding to the transducer of which the defect detection is completed, among the plurality of transducers.

At operation S171 the driving circuit stops operations corresponding to at least one defective transducer among the plurality of transducers. According to an embodiment, the operation S171 may include an operation of outputting a voltage having a preset level corresponding to at least one transducer detected as defective by the detection circuit by the voltage setting circuit provided respectively with respect to the plurality of driving circuits. The voltage setting circuit may include a switch for controlling the output of the voltage having the preset level.

According to an embodiment, there may be added an operation of controlling a supply of power based on a proportion of driving circuits, stopped by the defect detection results of the detection circuit, among the plurality of driving circuits.

According to an embodiment, there may also be added an operation of displaying the defect detection results of the detection circuit with regard to each of the plurality of transducers on the display. Alternatively, there may be added an operation of transmitting the defect detection results of the detection circuit with regard to each of the plurality of transducers to the display apparatus.

According to an embodiment, there also may be added an operation of detecting whether the plurality of transducers is defective by sequentially connecting the driving circuits through the detection circuit provided as a single circuit corresponding to the plurality of driving circuits. Alternatively, the plurality of driving circuits itself may be used to detect whether any of the plurality of transducers is defective. In this case, the plurality of driving circuits may include a comparator to detect whether any of the plurality of transducers is defective.

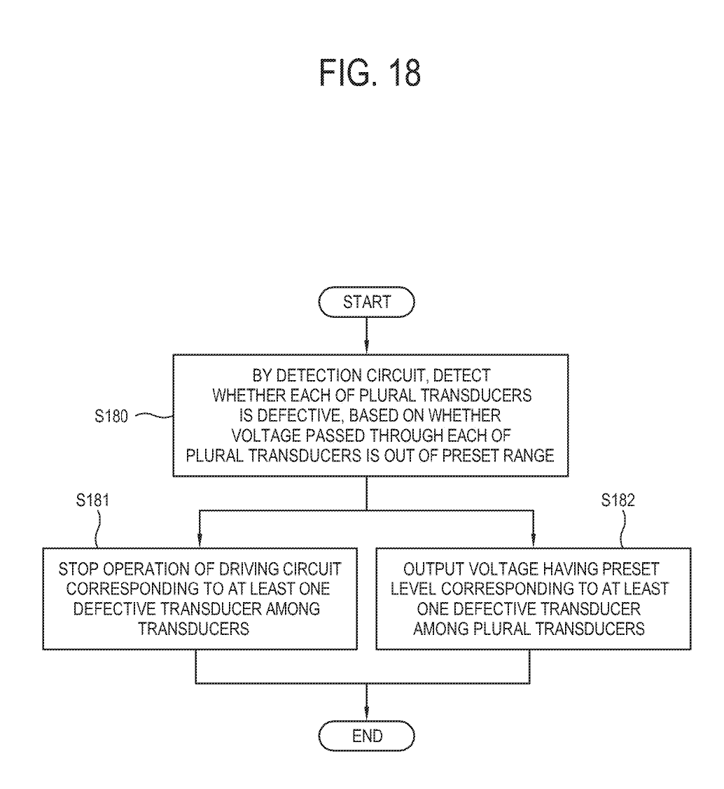

FIG. 18 is a flowchart of a method of controlling an ultrasonic image apparatus according to an embodiment of the present disclosure.

Referring to FIG. 18, at operation S180 it is determined by the detection circuit whether any of the plurality of transducers is defective based on whether a voltage passed through one or more of the plurality of transducers is out of a preset range. At operation S181, a driving circuit stops operations corresponding to at least one defective transducer among the plurality of transducers, and at the same time at operation S182, a voltage having a preset level is output corresponding to at least one defective transducer among the plurality of transducers.

In the ultrasonic image apparatus according to an embodiment, there is a determination whether any of the plurality of transducers is defective before the ultrasonic measurement and thus, the defective transducers are inactivated before the ultrasonic measurement thereby ensuring safety. Further, it is possible to prevent quality of an image from being deteriorated due to the defective transducers.

As described above, there is a determination whether any of a plurality of transducers is defective before the ultrasonic image apparatus performs ultrasonic measurement, thereby further ensuring reliability in the use of the transducers.

Further, the ultrasonic image apparatus inactivates the defective transducers among the plurality of transducers and/or inactivates the device before the ultrasonic image apparatus performs ultrasonic measurement, thereby further ensuring safety.

While the present disclosure has been shown and described with reference to various embodiments thereof, it will be understood by those skilled in the art that various changes in form and detail may be made therein without departing from the spirit and scope of the present disclosure as defined by the appended claims and their equivalents.

What is claimed is:

1. An ultrasonic image apparatus comprising:
a plurality of transducers configured to output an ultrasonic signal and receive a feedback signal;
a driver comprising a plurality of driving circuits respectively connected to the plurality of transducers, each driving circuit configured to output the ultrasonic signal and receive the feedback signal;
a detection circuit respectively corresponding to the plurality of driving circuits, the detection circuit configured to detect whether any of the plurality of transducers is defective; and
at least one processor configured to control the driving circuit to stop operations corresponding to at least one defective transducer among the plurality of transducers based on a defect detection result from the detection circuit with regard to the plurality of transducers.

2. The ultrasonic image apparatus according to claim 1, wherein the detection circuit is further configured to detect whether any of the plurality of transducers is defective based on whether a voltage passed through one or more of the plurality of transducers is out of a preset range.

3. The ultrasonic image apparatus according to claim 1, wherein the detection circuit comprises:
a low-voltage current source; and
a comparator.

4. The ultrasonic image apparatus according to claim 1, wherein the at least one processor is further configured to control the detection circuit to stop operations corresponding to a transducer of which a defect detection is completed, among the plurality of transducers.

5. The ultrasonic image apparatus according to claim 1, further comprising:
a voltage setting circuit corresponding to each of the plurality of driving circuits,
wherein the voltage setting circuit is configured to output a voltage having a preset level corresponding to at least one transducer detected as defective by the detection circuit.

6. The ultrasonic image apparatus according to claim 5, wherein the voltage setting circuit comprises a switch configured to selectively control an output of a voltage having a preset level.

7. The ultrasonic image apparatus according to claim 1, wherein the at least one processor is further configured to control a supply of power based on a proportion of driving circuits stopped in accordance with defect detection results from the detection circuit, among the plurality of driving circuits.

8. The ultrasonic image apparatus according to claim 1, further comprising:
a display,
wherein the at least one processor is further configured to control the display to display a defect detection result from the detection circuit with regard to each of the plurality of transducers.

9. The ultrasonic image apparatus according to claim 1, further comprising:
a communicator configured to connect with a display apparatus,
wherein the at least one processor is further configured to control the communicator to transmit a defect detection result from the detection circuit with regard to each of the plurality of transducers to the display apparatus.

10. The ultrasonic image apparatus according to claim 1,
wherein the detection circuit is provided as a single circuit corresponding to the plurality of driving circuits, and
wherein the detection circuit is further configured to connect with each of the plurality of driving circuits in sequence to detect whether any of the plurality of transducers is defective.

11. The ultrasonic image apparatus according to claim 1, wherein one or more of the plurality of driving circuits itself is used to detect whether any of the plurality of transducers is defective.

12. The ultrasonic image apparatus according to claim 11, wherein the plurality of driving circuits comprises a comparator for detecting whether any of the plurality of transducers is defective.

13. A method of controlling an ultrasonic image apparatus, the method comprising:
detecting whether any of a plurality of transducers is defective through a detection circuit provided in each of a plurality of driving circuits respectively connected to the plurality of transducers, each driving circuit configured to output an ultrasonic signal and receive a feedback signal; and
stopping an operation of a driving circuit corresponding to at least one defective transducer among the plurality of transducers based on a defect detection result from the detection circuit with regard to the plurality of transducers.

14. The method according to claim 13, wherein the detecting of whether any of the plurality of transducers is defective comprises detecting whether any of the plurality of transducers is defective based on whether a voltage passed through one or more of the plurality of transducers is out of a preset range.

15. The method according to claim 13, wherein the detection circuit comprises:
a low-voltage current source; and
a comparator.

16. The method according to claim 13, further comprising stopping an operation of the detection circuit corresponding to a transducer of which a defect detection is completed, among the plurality of transducers.

17. The method according to claim 13, wherein the stopping of the operation of the driving circuit comprises outputting a voltage having a preset level corresponding to at least one transducer detected as defective in the detection circuit, by a voltage setting circuit provided corresponding to each of the plurality of driving circuits.

18. The method according to claim 17, wherein the voltage setting circuit comprises a switch for selectively controlling an output of a voltage having a preset level.

19. The method according to claim 13, further comprising controlling a supply of power based on a proportion of driving circuits stopped in accordance with defect detection results from the detection circuit, among the plurality of driving circuits.

20. The method according to claim 13, further comprising displaying a defect detection result from the detection circuit with regard to each of the plurality of transducers.
